(19) United States
(12) Patent Application Publication
(10) Pub. No.: US 2012/0041462 A1
(43) Pub. Date: Feb. 16, 2012

(54) APPARATUS FOR FORMING HOLE IN SPONGELIKE BONE

(75) Inventors: Sung-Ho Maeng, Incheon (KR); Chang-Yong Kim, Gyeonggi-do (KR)

(73) Assignee: SAE WON MEDITECH. CO., LTD., Siheung-si, Gyeonggi-do (KR)

(21) Appl. No.: 13/203,292

(22) PCT Filed: Feb. 4, 2010
(86) PCT No.: PCT/KR2010/000701

§ 371 Date: Nov. 2, 2011

(30) Foreign Application Priority Data
Feb. 27, 2009 (KR) 10-2009-0017045
May 20, 2009 (KR) 10-2009-0044155
Oct. 23, 2009 (KR) 10-2009-0101369

(51) Int. Cl. A61B 17/34; A61M 29/00

(52) U.S. Cl. 606/185; 606/192

(57) ABSTRACT

Disclosed herein is an apparatus for forming a hole in a spongelike bone. The apparatus includes a catheter tube, an expansible structure coupled to a front end of the catheter tube, a hub having a fitting coupled to a rear end of the catheter tube, and a probe. The catheter tube includes the probe and one lumen surrounding the probe, both of which are disposed inside the catheter tube. The probe is fixed to the hub or a rear cap of the hub at a rear end thereof and comes into contact with an inner front end of the expansible structure at a front end thereof.

### TECHNICAL FIELD

The present invention relates generally to an apparatus for forming a hole in a spongelike bone and, more particularly, to a structure of a catheter and an expansible structure that is coupled to an upper portion of the catheter structure.

### BACKGROUND ART

A spinal curvature restoration procedure is an operating procedure wherein a damaged vertebra is restored to its original height and angle in a kyphosis, and then a bone filler is injected into the damaged vertebra to stabilize it. This operation is generally performed percutaneously, and the restoration of the height and angle of the vertebra is done by intravertebral expansion based on the pressure of liquid or a mechanical method up to now.

A typical apparatus used in the spinal curvature restoration procedure is made up of a needle and a wire pin, a cannula and an expander, and a cement filler and a pusher. Here, depending on whether or not a balloon catheter is used, spinal curvature restoration procedures are divided into a typical restoration procedure and a balloon restoration procedure.

Making reference to the balloon restoration procedure in detail, an elongated special tube is inserted into a compression curvature region. A balloon is inserted into the region through the tube, and then is expanded up to a normal height of the vertebra. Then, the balloon is removed, and the hole formed by the removal of the balloon is filled with bone cement.

An example of the apparatus for forming the hole used in the above-mentioned procedure is disclosed in Korean Patent No. 10-0793005. As shown in FIG. 1, such an apparatus 76 includes: a catheter tube 78 that has a proximal end 80, a distal end 82, a first lumen 88, and a second lumen 94; an expansible structure 86 such as a balloon that is supported on the distal end of the catheter 78, has a tip, and communicates with the first lumen 88 of the catheter 78; and a probe 96 that is removably inserted into the second lumen of the catheter tube 78. The second lumen 94 in a tool 76 that is extended to the tip of the expansible structure 86 through the first lumen 88 of the catheter tube.

However, since the tool 76 forming the hole includes the first lumen 88, the second lumen 94, and the probe 96, it has a very complicated structure that makes the cost of production high. Further, since the probe 96 made of metal extends to the tip of the expansible structure 86, there is a fear that bringing the metal probe into contact with a human body will cause problems when the expansible structure 86 bursts. Thus, a method capable of solving this problem is being sought out.

Further, as shown in FIG. 7, the expansible structure (e.g. the balloon) that has been generally used in the related art is formed of an elastomer having a single composition, and there is always a chance that it will burst during an operation. When the expansible structure (e.g. the balloon) is burst, a fragment of the expansible structure (e.g. the balloon) may remain in the bone without being recollected in addition to the problem that the metal probe is brought into contact with the human body. As a result, the operation may be delayed, which increases the pain of a patient. Accordingly, a study into improving this problem is required.

### DISCLOSURE

### Technical Problem

Accordingly, the present invention has been made keeping in mind the above problems occurring in the prior art, and an objective of the present invention is to provide an apparatus for forming a hole in a spongelike bone, which simplifies the structure of a conventional apparatus for forming a hole in a spongelike bone with its functionality maintained without a change, and which is a very economical apparatus.

Another objective of the present invention is to provide an apparatus for forming a hole in a spongelike bone, in which the diameter of a catheter tube is remarkably reduced by the structural simplification as described above.

Yet another objective of the present invention is to provide an expansible structure (e.g. a balloon) which improves a conventional expansible structure having a mono-layer structure so as to have a multi-layer structure including a reticular structure, thereby minimizing a bursting possibility, and which is used to form a hole in a human body in which the position and expanded shape of the balloon can be monitored without using a marker of platinum and an expensive contrast medium depending on a material of the reticular structure.

### Technical Solution

In order to accomplish the above objectives, the present invention provides an apparatus for forming a hole in a spongelike bone, which includes: a catheter tube; an expansible structure coupled to a front end of the catheter tube; a hub having a fitting coupled to a rear end of the catheter tube; and a probe, wherein the catheter tube includes the probe and one lumen surrounding the probe, both of which are disposed inside the catheter tube, and the probe is fixed to the hub or a rear cap of the hub at a rear end thereof and comes into contact with an inner front end of the expansible structure at a front end thereof.

Here, the front end of the catheter tube may extend to the inner front end of the expansible structure, and the extension part of the catheter tube may include at least one fluid inflow and outflow hole.

Further, the expansible structure may have a balloon shape and be formed by coating a reticular structure formed of a polymer or metal material with a polymer.

In order to accomplish the above objectives, the present invention provides an expansible structure used in an apparatus for forming a hole in a spongelike bone, which has a balloon shape and is formed by coating a reticular structure formed of a polymer or metal material with a polymer.

### Advantageous Effects

According to the present invention, the apparatus for forming a hole in a spongelike bone is improved and made into a simple structure by removing a tube structure forming a conventional second lumen, thereby providing easy production, convenient usage, and high economical benefits.

Further, the diameter of the catheter tube is reduced by the structural simplification as described above, and thus an insertion hole which is formed in a human body as part of an operation to make way for the apparatus can be reduced in size. Thus, the effect of reducing the pain of a patient and the effect of facilitating the operation are provided.

Further, the catheter tube extends to the inner front end of the expansible structure (e.g. the balloon), and the fluid inflow and outflow hole is formed in the extension part of the catheter tube. Thereby, it is possible to feed and recollect fluid into and from the expansible structure in a rapid, easy manner. Thus, the inventive apparatus provides the effects of cutting back on operating time and facilitating the operation.

According to the present invention, the expansible structure is improved so as to have a multi-layer structure of the reticular structure formed of a polymer or metal material and the polymer coating the reticular structure, so that it is allowed to minimize a possibility of being burst in the human body. Further, when the reticular structure is formed of a metal material, the reticular structure itself is displayed on an X-ray monitor. As such, it is easy to check position and expanded shape of the balloon, and thus a higher precision operation is possible. Further, since it is unnecessary to separately use the marker made of platinum and the expensive contrast medium, the expansible structure is very economical.

### DESCRIPTION OF DRAWINGS

FIGS. 1 and 2 show a conventional apparatus for forming a hole in a spongelike bone;

FIG. 3 shows an apparatus for forming a hole in a spongelike bone according to an embodiment of the present invention;

FIG. 4 shows a probe integrally formed with a hub in the apparatus for forming a hole in a spongelike bone according to the embodiment of the present invention;

FIG. 5 shows an extension part of a catheter tube in the apparatus for forming a hole in a spongelike bone according to the embodiment of the present invention;

FIG. 6 shows the extension part of the catheter tube and the probe integrally formed with the hub in the apparatus for forming a hole in a spongelike bone according to the embodiment of the present invention;

FIG. 7 shows a conventional expansible structure having only elastomer layer used to form the hole in a human body, wherein FIG. 7(a) is for the expansible structure before expansion and FIG. 7(b) is for the expansible structure after expansion;

FIG. 8 shows a reticular structure constituting an inventive expansible structure used to form the hole in the human body;

FIG. 9 is a photograph of the reticular structure constituting the inventive expansible structure used to form the hole in the human body, wherein FIG. 9(a) is for the reticular structure knitted with fibriform polymer wires, and FIG. 9(b) is for the reticular structure knitted with metal wires;

FIG. 10 shows the metal wire having a tensile spring shape forming the reticular structure used in the inventive expansible structure, wherein FIG. 10(a) is of the metal wire before expansion, and FIG. 10(b) is of the metal wire after expansion;

FIG. 11 shows the inventive expansible structure used to form the hole in the human body, wherein FIG. 11(a) is the expansible structure before expansion, and FIG. 11(b) is the expansible structure after expansion; and

FIG. 12 shows a partial cross section of the inventive expansible structure.

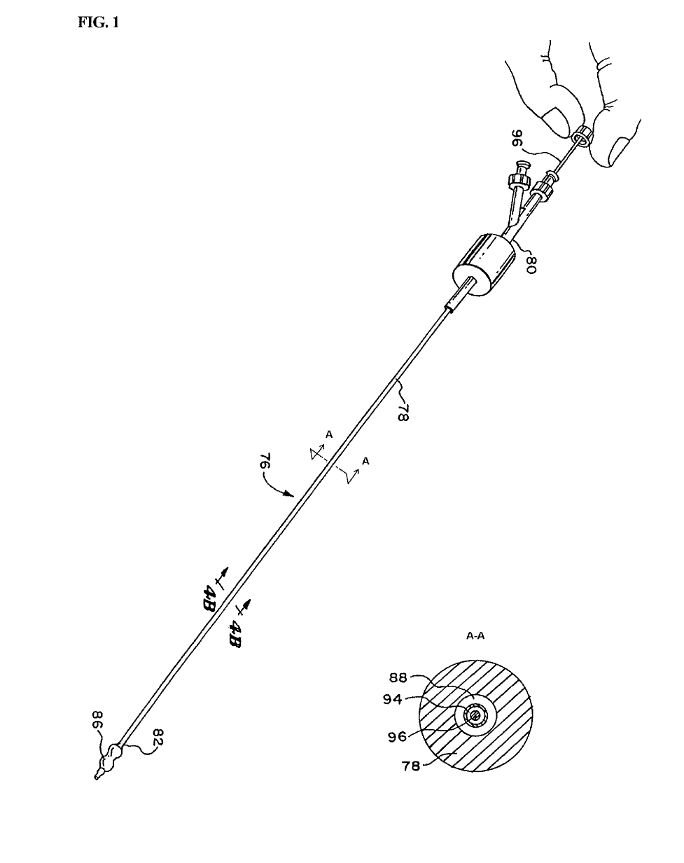

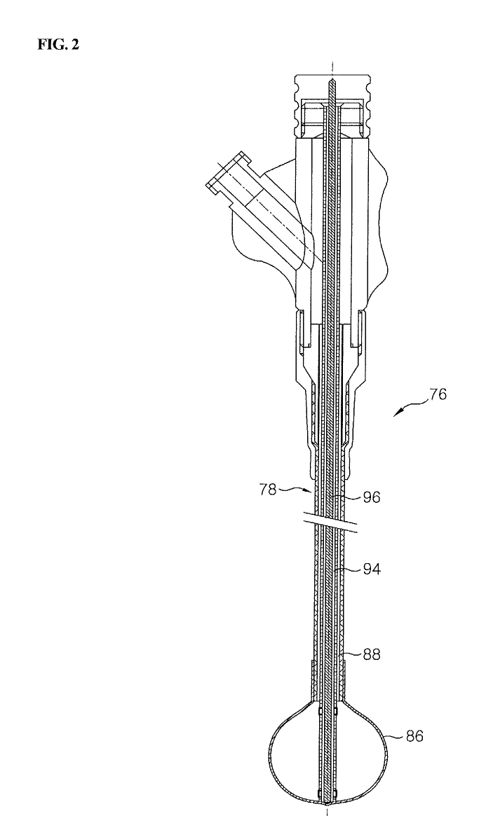

### BRIEF DESCRIPTION OF SYMBOLS USED IN DRAWINGS

| | |
|---|---|
| 10: catheter tube | 11: lumen |
| 12: extension part of catheter tube | 13: fluid inflow and outflow hole |
| 14: fibriform polymer or metal wire | |
| 15: expansion allowance | 16: metal wire having tensile |
| | spring shape |
| 20: probe (or support rod) | 21: marker |
| 30: hub | 31: fitting |
| 40: hub rear cap | 50: expansible structure |
| 51: reticular structure | 52: polymer for coating |
| 100: apparatus for forming hole in | |
| spongelike bone | |

### BEST MODE

Reference now should be made to the different drawings, throughout which the same reference numerals are used to designate the same or similar components. The detailed descriptions of known functions and constructions unnecessarily obscuring the subject matter of the present invention will be avoided below.

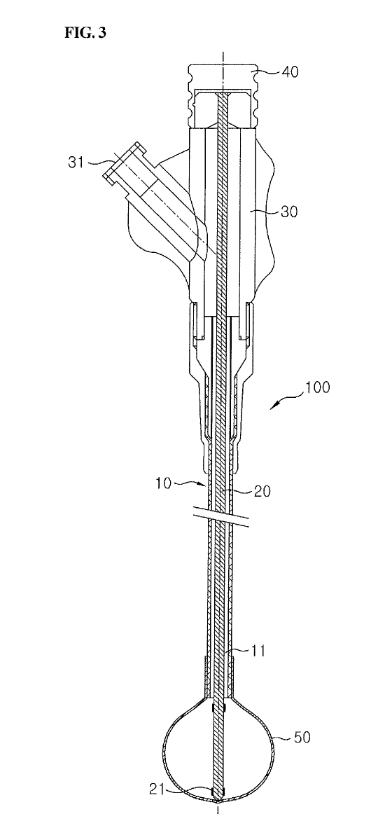

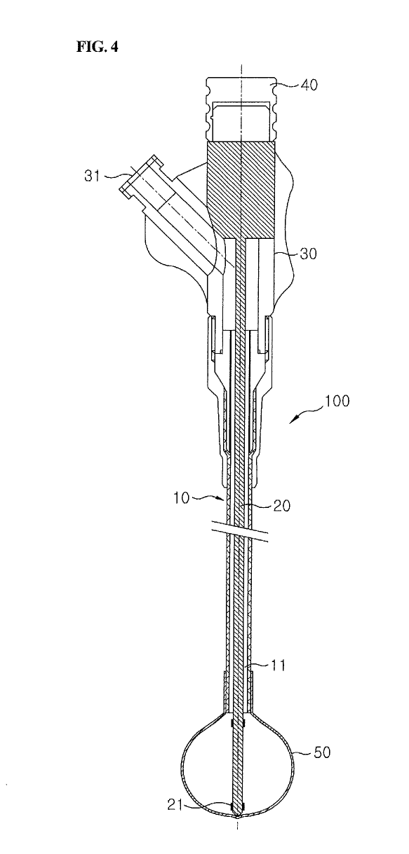

As shown in FIGS. 3 and 4, the present invention is directed to an apparatus 100 for forming a hole in a spongelike bone, which includes a catheter tube 10, an expansible structure 50 coupled to a front end of the catheter tube, a hub 30 having a fitting 31 coupled to a rear end of the catheter tube, and a probe 20. The catheter tube 10 is made up of the probe (also called a “support rod”) 20 and one lumen 11 surrounding the probe, both of which are disposed inside the catheter tube. The probe 20 is fixed to the hub 30 or a rear cap 40 of the hub at a rear end thereof, and comes into contact with an inner front end of the expansible structure 50 at a front end thereof.

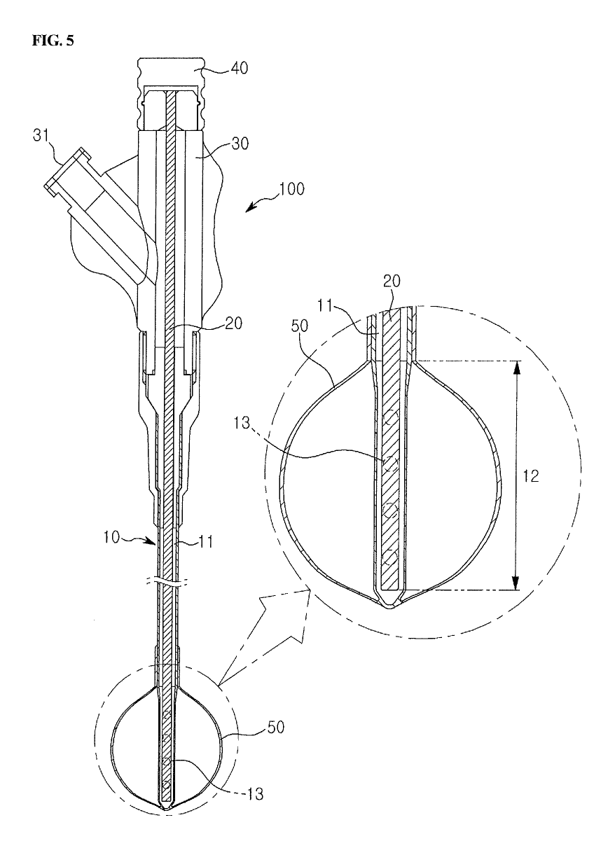

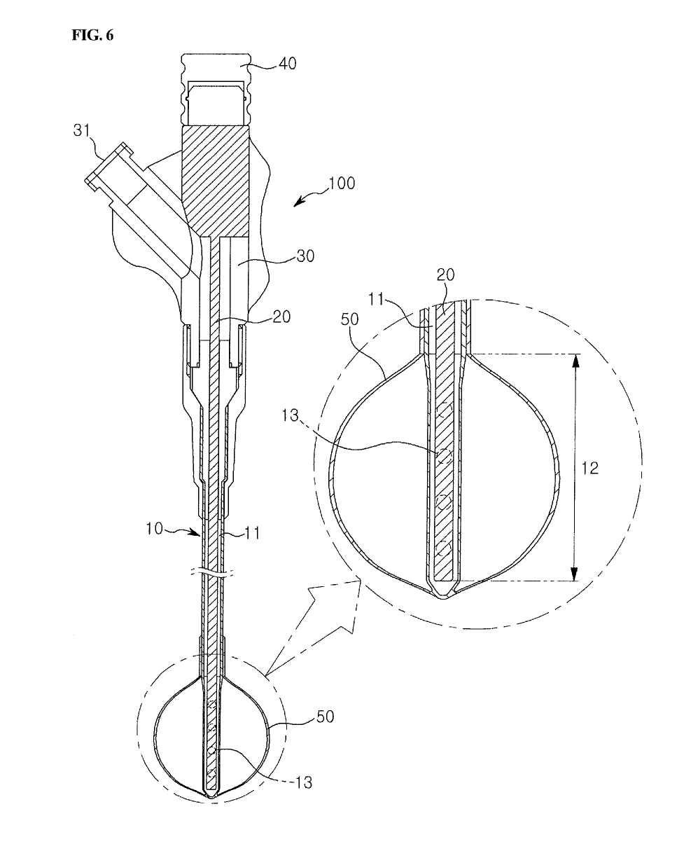

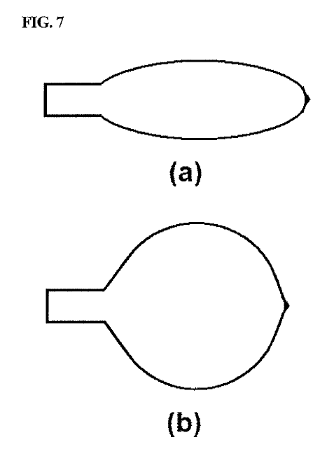

Further, as shown in FIGS. 5 and 6, the present invention is directed to an apparatus 100 for forming a hole in a spongelike bone, in which the front end of the catheter tube extends to the inner front end of the expansible structure 50, and the extension part 12 of the catheter tube has at least one fluid inflow and outflow hole 13.

In the present invention, since the catheter tube 10 is provided therein with only one lumen 11, it is possible for the catheter tube 10 to be configured to have a smaller diameter than a conventional catheter tube. Thus, a hole formed in a human body for the purpose of the operation can be reduced in diameter, so that the catheter tube 10 can provide the effect of reducing the pain of a patient and the effect of getting the patient to rapidly recover following the operation.

The extension part 12 of the catheter tube may be formed in a structure where a diameter thereof is smaller than that of the catheter tube 10 from the hub 30 to a portion coupled with the expansible structure 50. The structure of the catheter tube extension part 12 facilitates the insertion of the expansible structure 50 into an operating region in the event of an operation.

The desired number of fluid inflow and outflow holes 13 formed in the catheter tube extension part 12 (FIG. 5) may be selected after taking into consideration the flow rate of fluid that flows in and out through the lumen 11 inside the catheter tube 10. The fluid inflow and outflow hole 13 is not substantially limited to its shape as long as it does not hinder the fluid from flowing in and out. When the fluid inflow and outflow hole 13 is formed at an end of the extension part 12, this provides the effect of more easily recollecting the fluid contained in the inner front end of the expansible structure 50.

A front end of the catheter tube extension part 12 may be formed to be blocked.

The catheter tube extension part 12 may be attached with at least one marker 21, the position of which can be detected, for instance, by X-rays. For example, the markers 21 may be attached to respective portions of the catheter tube extension part 12 which are located at an entrance portion and the front end of the expansible structure 50.

As a material of the catheter tube 10, any material may be used as long as the catheter tube can move forward through the cannula instrument. For example, the catheter tube may be made using a surgical grade plastic material having excellent flexibility, such as vinyl, nylon, polyethylene, ionomer, polyurethane, polyethyleneterephthalate (PET), or the like. Further, a harder material may be selected to enhance rigidity and manipulability.

The lumen formed in the catheter tube 10 communicates with the expansible structure 50 and the fitting 31. The fitting 31 serves to connect the lumen to a fluid source, for instance, a sterilized salt water source or a radiopaque contrast medium source.

The probe 20 is fixed to the rear cap 40 of the rear end of the hub 30, and is inserted through a passage in the hub 30 and the lumen 11 inside the catheter tube to the front end of the expansible structure 50. If necessary, the probe 20 may be formed in a separable structure.

As shown in FIGS. 4 and 6, the probe 20 may, however, be integrally formed with the hub 30 or fixed to the hub 30 by an adhering method.

As material for the probe 20, metal materials or polymers such as plastics which are available for medical use may be used without restriction. Among them, the material of the probe 20 may be selected in consideration of strength required for the probe 20, physical properties required for integration with the hub 30, adhesion to the hub 30, and so on. It is particularly preferable that the probe 20 be made of polymer, because it is possible to avoid problems that are caused by direct contact of the probe of metal with the human body even if the expansible structure 50 were to burst.

The probe 20 functions to keep the expansible structure 50 linear at a distal end of the catheter until the catheter 100 runs through the cannula instrument to reach a target tissue region. Once the expansible structure 50 leaves the cannula instrument to be placed inside the bone, the probe 20 can be extracted. Thereby, it is possible to increase flexibility of the catheter tube 10, and to easily manipulate the expansible structure 50 in the bone.

The probe 20 may have a linear shape, or another shape where its end region can be bent at a desired point of time by providing a previously formed memory. In the case of providing the memory, the probe 20 is in the linear shape by overcoming the memory when kept in the cannula instrument. As the probe 20 leaves the cannula instrument to move into a target region, the end region of the probe 20 is bent by the previously formed memory, so that a major axis of the expansible structure 50 is shifted. In this case, a direction of the expansible structure 50 may be changed by the probe 20 that is previously bent in the expansible structure 50, and the anatomical alignment with the target region is made more excellent.

A part of the probe 20, which extends to the inside of the expansible structure 50, may be attached with at least one marker 21, the position of which can be detected, for instance, by X-rays. For example, the markers 21 may be attached to respective portions that are located at the entrance portion and the front end of the expansible structure 50, within the part of the probe 20 which extends to the inside of the expansible structure 50.

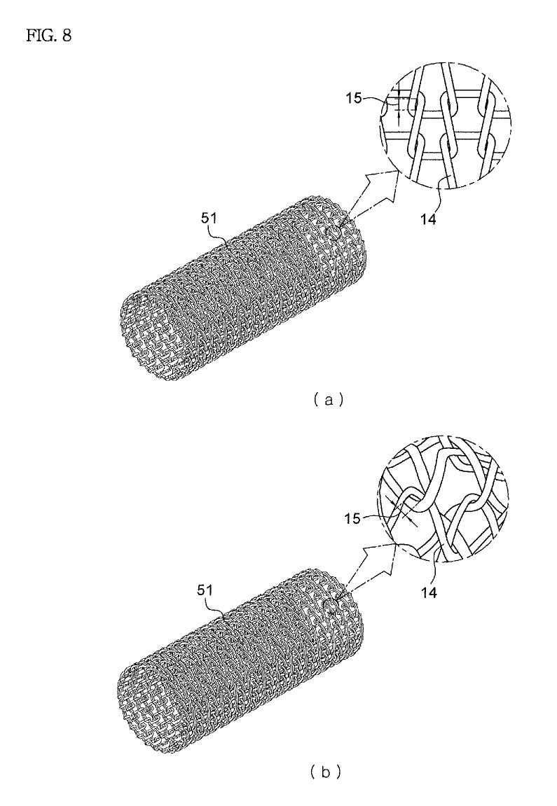

For the apparatus 100 for forming a hole in a spongelike bone according to the present invention, a variety of types of expansible structures 50 may be used. Particularly, the expansible structure 50 having a balloon shape may be used. As the balloon-shaped expansible structure 50, ones known in the related art may be used without restriction. However, to reduce the possibility of being burst in the human body, one formed by coating a reticular structure 51 formed of polymer or metal with a polymer 52, as shown in FIG. 8 can be used. When the reticular structure 51 is formed of metal, the reticular structure itself is displayed on an X-ray monitor. As such, it is easy to check the position and expanded shape of the balloon, and thus a higher precision operation is possible. Further, due to this advantage, it is unnecessary to separately use the marker made of platinum and the expensive contrast medium, so that economical benefits are provided. The expansible structure 50 will be described below in greater detail.

Further, as shown in FIGS. 8 through 12, the present invention is directed to the expansible structure 50 used in the apparatus 100 for forming a hole in a spongelike bone, in which the polymer 52 is coated on the reticular structure 51 formed of polymer or metal.

In the present invention, the reticular structure 51 refers to a structure in which a plurality of holes are formed by a crossed polymer or metal material regardless of a structure, and should be genetically interpreted. For example, the reticular structure 51 may include a structure manufactured by crossing several fibriform polymer or metal wires, or a structure manufactured by knitting the fibriform polymer or metal wires in the form of a knitted fabric.

However, in the present invention, since the reticular structure formed of the polymer or metal material should be expanded when the balloon is expanded, it is preferable to use the reticular structure manufactured by knitting the fibriform polymer or metal wires in the form of a knitted fabric, as shown in FIG. 8. This is because, when the reticular structure is knitted with the fibriform polymer or metal wires, expansion allowances 15, which allow the reticular structure 51 to be expanded when the balloon is expanded, are generated between loops, so that the balloon can expand smoothly.

Here, the method used to adjust the expansion allowances 15 may be a knitting method.

FIGS. 8(a) and 8(b) show tubular reticular structures 51 knitted by different methods. It can be seen from the state where the fibriform polymer or metal wires 14 are knitted to form the reticular structure 51 that the expansion allowances 15, which allow the reticular structure 51 to be expanded when the expansible structure 50 is expanded, are formed between the loops.

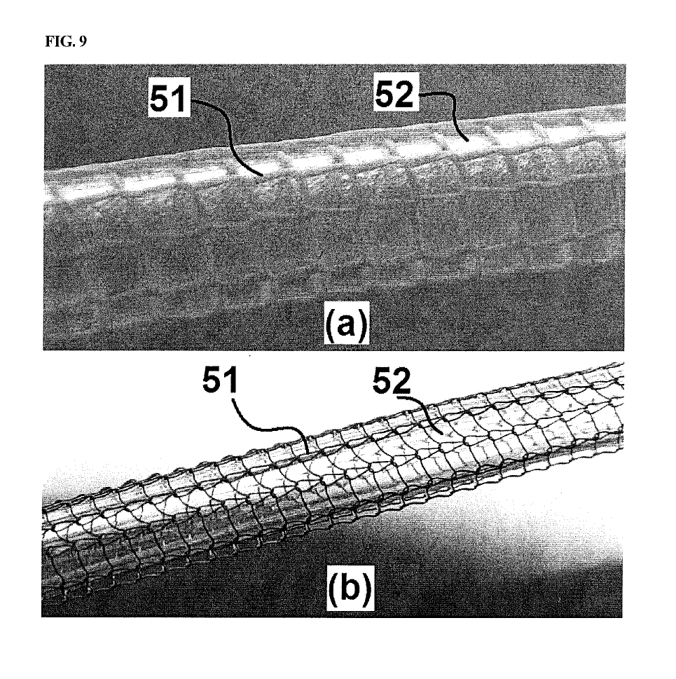

A photograph shown in FIG. 9 is an actual photograph of the reticular structure 51 used in the present invention. FIG. 9(a) is a photograph of the reticular structure 51 formed of the fibriform polymer, and FIG. 9(b) is a photograph of the reticular structure 51 knitted with the metal wires. In the photographs, a member located below the reticular structure 51 is a polymer 52 tube for coating, and is used to coat the inside of the reticular structure 51 when the reticular structure 51 is formed in a balloon shape.

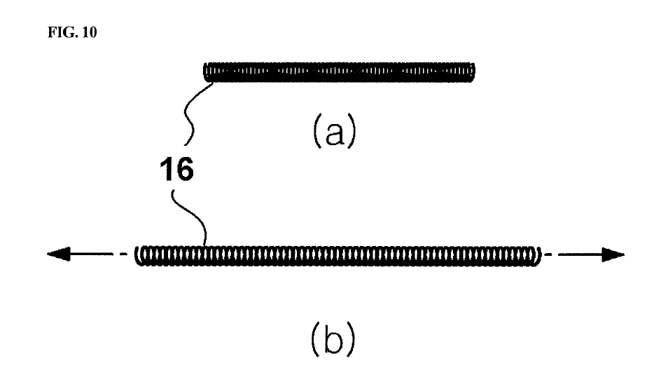

In the present invention, when the reticular structure 51 is formed of a metal material, the metal wires forming the reticular structure preferably have the shape of a tensile spring 16, as shown in FIG. 10. This is because it is possible to secure the expansion allowances 15 by the knitting method as described above, but when the metal wires having the shape of the tensile spring 16 are used, the reticular structure may be more smoothly expanded and contracted when the balloon is expanded and removed. Thus, when the reticular structure 51 is manufactured using the metal wires having the shape of the tensile spring 16, there is no fear of the expansible structure 50 including such a reticular structure 51 being destroyed during the operation. Further, since the reticular structure itself is displayed on the X-ray monitor, it is easy to check the position and expanded shape of the expansible structure 50, and thus a higher precision operation is possible. Further, since the expansible structure 50 is contracted and reduced in volume when removed, it is easily removed.

In the present invention, the kind of metal material used for the reticular structure 51 is not substantially restricted. However, since the metal material is used in the human body, it is preferable to use a material suitable for medical use such as stainless steel.

A diameter of the metal wire used as the metal material is not substantially restricted. However, the diameter of the metal wire should be suitable to form the expansible structure. For example, the diameter of the metal wire may range from about 0.001 mm to about 0.1 mm. Further, when the metal wire has the tensile spring shape, the diameter of the metal wire may range from about 0.005 mm to about 0.3 mm.

In the present invention, the reticular structure 51 may be knitted with one strand of the fibriform polymer or metal wires or two or more strands of the fibriform polymer or metal wires. Here, to manufacture the reticular structure in a firm structure, the reticular structure is preferably knitted with one strand of the fibriform polymer or metal wires.

In the expansible structure 50 of the present invention, a kind of the polymer that is used to form the reticular structure 51 is not substantially restricted. However, a polymer having excellent strength and elongation is preferable. For example, elastomer, Peek®, silicon, latex, or the like may be used. The elastomer may include nylon, polyurethane, polyethylene, polypropylene, polystyrene, and so on. Among them, the Peek® is a polymer material that has been proven to not be harmful to the human body and that is thought to be one of thermoplastic materials exhibiting the highest functionality, and is currently used as an implant material in connection with a spine (see the website http://www.victrex.com). Particularly, as the polymer material used to form the reticular structure 51, nylon, Peek®, polyurethane, or the like may be used.

In the present invention, as the polymer 52 that coats and seals the reticular structure, a typical material used in the related art may be used without a restriction, and a polymer having excellent strength and elongation is preferable. For example, elastomer, Peek®, silicon, latex, or the like may be used. The elastomer may include nylon, polyurethane, polyethylene, polypropylene, polystyrene, and so on.

In the present invention, as the polymer used to form the reticular structure 51 and the polymer 52 used to coat and seal the reticular structure 51, a homogeneous or heterogeneous polymer may be used.

In the present invention, when the reticular structure 51 is formed of the polymer, a ratio of tensile strength of the polymer 52 for coating the reticular structure to that of the polymer used to form the reticular structure 51 is 1:0.5 to 1:10, preferably 1:1.0 to 1:3.0. If the tensile strength of the polymer used to form the reticular structure 51 is 0.5 times less than that of the polymer 52 for coating the reticular structure, it is difficult to sufficiently obtain the desired effects. In contrast, if the tensile strength of the polymer used to form the reticular structure 51 is 10 times more than that of the polymer 52 for coating the reticular structure, only the polymer 52 for coating the reticular structure is elongated (expanded), and thus there is a chance of increasing the danger of bursting. Thus, the tensile strength of the polymer 52 for coating the reticular structure and the tensile strength of the polymer used to form the reticular structure 51 are selected in consideration of the characteristics of the respective polymers, and preferably are within a proper range on the basis of the predicted behaviors of the respective polymers when the balloon is expanded.

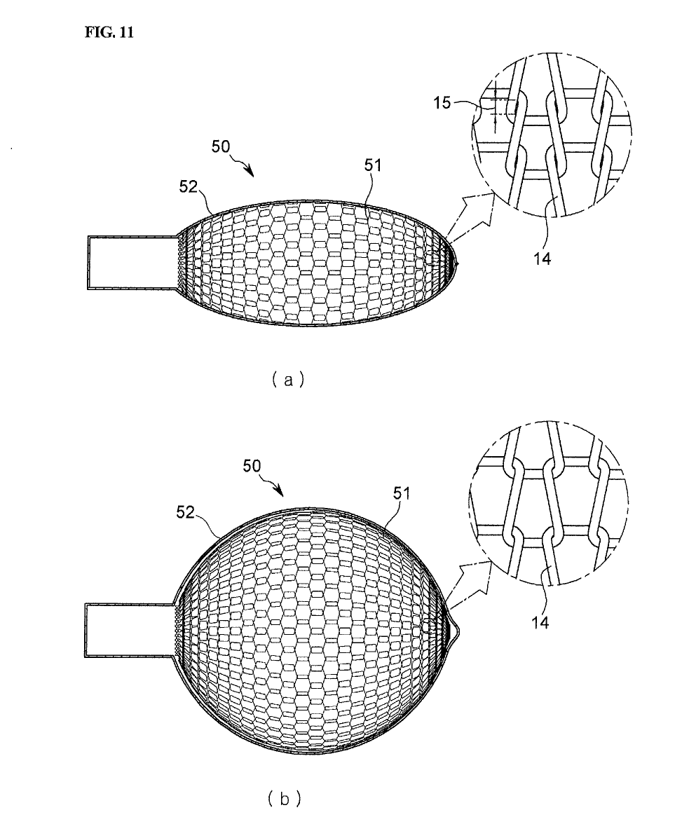

FIG. 11 shows an example of the expansible structure 50 of the present invention, wherein FIG. 11(a) shows the shape before expansion, and FIG. 11(b) shows the shape after expansion. In FIG. 11(a), the expansion allowances 15 allow the reticular structure 51 to be expanded so that the balloon is expanded to a predetermined size when fluid flows into the expansible structure 50.

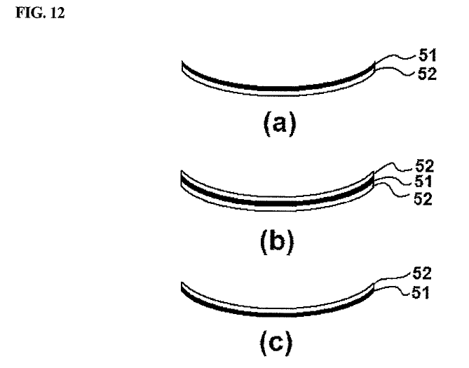

In the expansible structure 50 of the present invention, a laminated structure in which the polymer 52 for coating the reticular structure is coated on the reticular structure 51 formed of the polymer or metal material is as shown in FIG. 12. In detail, the laminated structure may include a structure in which the polymer 52 for coating the reticular structure is coated on an outer surface of the reticular structure 51 as in FIG. 12(a), a structure in which the polymer 52 for coating the reticular structure is coated on outer and inner surfaces of the reticular structure 51 to form a sandwich structure as in FIG. 12(b), and a structure in which the polymer 52 for coating the reticular structure is coated on an inner surface of the reticular structure 51 as in FIG. 12(c).

The method of manufacturing the expansible structure 50 of the present invention is not substantially restricted. For example, a known method of manufacturing a hose including a reticular structure may be used for reference. For example, the reticular structure is primarily knitted with the fibriform polymer or metal wires on an outer surface of a tube, which has been primarily extruded using a polymer material such as elastomer, using a knitting machine (see FIG. 9). Then, the knitted reticular structure is secondarily coated with a polymer material such as elastomer, and is cut into a proper size and formed into balloon shape. Thereby, the expansible structure 50 of the present invention is finished.

The inventive balloon used to form a hole in a human body may be applied to various fields. For example, the inventive balloon may be very useful for a balloon catheter used for the balloon treatment of vertebral compression fractures.

Although the embodiments of the present invention have been disclosed for illustrative purposes, those skilled in the art will appreciate that various modifications, additions and substitutions are possible, without departing from the scope and spirit of the invention as disclosed in the accompanying claims.

1. An apparatus for forming a hole in a spongelike bone, comprising:
a catheter tube;
an expansible structure coupled to a front end of the catheter tube;
a hub having a fitting coupled to a rear end of the catheter tube; and
a probe,
wherein the catheter tube includes the probe and one lumen surrounding the probe, both of which are disposed inside the catheter tube, and the probe is fixed to the hub or a rear cap of the hub at a rear end thereof and comes into contact with an inner front end of the expansible structure at a front end thereof.

2. The apparatus as set forth in claim 1, wherein the front end of the catheter tube extends to the inner front end of the expansible structure, and the extension part of the catheter tube includes at least one fluid inflow and outflow hole.

3. The apparatus as set forth in claim 2, wherein the extension part of the catheter tube has a diameter smaller than that of the catheter tube running from the hub to a portion coupled with the expansible structure.

4. The apparatus as set forth in claim 2, wherein the extension part of the catheter tube is closed at a front end thereof.

5. The apparatus as set forth in claim 1, wherein the probe is formed of a polymer suitable for medical use, and is integrally formed with the hub or fixed to the hub by an adhering method.

6. The apparatus as set forth in claim 1, wherein the expansible structure has a balloon shape, and is formed by coating a polymer on a reticular structure formed of a polymer or metal material.

7. An expansible structure used in an apparatus for forming a hole in a spongelike bone, which has a balloon shape and is formed by coating a reticular structure formed of a polymer or metal material with a polymer.

8. The expansible structure as set forth in claim 7, wherein the reticular structure is a knitted fabric that is knitted with fibriform polymer or metal wires.

9. The expansible structure as set forth in claim 8, wherein the reticular structure is a knitted fabric that is knitted with one strand of the fibriform polymer or metal wires.

10. The expansible structure as set forth in claim 8, wherein the metal wires are formed in a shape of a tensile spring.

11. The expansible structure as set forth in claim 8, wherein the metal wires having the tensile spring shape are formed of stainless steel.

12. The expansible structure as set forth in claim 7, wherein, when the reticular structure is formed of a polymer, a ratio of tensile strength of the polymer for coating the reticular structure to that of the polymer used to form the reticular structure is 1:0.5 to 1:10.

13. The expansible structure as set forth in claim 7, wherein the reticular structure formed of the polymer or metal material has a sandwich structure in which inner and outer surfaces thereof are coated with a polymer.
